(19) United States
(12) Patent Application Publication
(10) Pub. No.: US 2026/0157874 A1
(43) Pub. Date: Jun. 11, 2026

(54) ROLL OF ANTI-MOUTH-BREATHING TAPE AND METHOD OF RESTRICTING BREATHING THROUGH A MOUTH OF A USER USING THE ROLL OF ANTI-MOUTH-BREATHING TAPE

(71) Applicant: Jared Christiansen, Lakeville, MN (US)

(72) Inventor: Jared Christiansen, Lakeville, MN (US)

(21) Appl. No.: 18/977,190

(22) Filed: Dec. 11, 2024

(51) Int. Cl. A61F 5/56

(52) U.S. Cl. CPC A61F 5/56

(57) ABSTRACT

A roll of anti-mouth-breathing tape includes a spool and a strip of anti-mouth-breathing tape. The spool includes a spindle and the strip is wound about the spindle.

The strip includes a substrate layer and an adhesive layer on the substrate layer. The strip is elongated. The strip includes a plurality of perforations oriented perpendicular to a length of the strip. Each of the perforations is spaced apart at intervals to form tape pieces that are selectively detachable from the strip when the strip is unwound. Each of the tape pieces is designed to be removably attached to lips of a user to restrict breathing through a mouth of the user and promote breathing through a nose of the user.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to tapes used to prevent mouth breathing, and more particularly pertains to a new roll of anti-mouth-breathing tape for easy dispensing of tape pieces. Anti-mouth-breathing tape is used to restrict or prevent breathing through a mouth of a user and promote breathing through a nose of the user. Such tape can be helpful in addressing snoring issues or just mouth breathing. The tape is placed on the lips of the user, which prevents the user from opening his or her mouth, thereby forcing breathing through the nose. However, the tape pieces are provided as individual units in a bag or box, which is inconvenient in size, especially for placement on a nightstand. In addition, each piece has backing material, which produces waste and can be a nuisance if the user is putting on a tape piece right before going to sleep, as the user must now pile up discarded backing material on the nightstand or get up out of bed to discard the backing material in a waste receptacle. Finally, each of the individual units is one size, which prevents any type of customization of the size of the tape pieces.

### (2) Description of Related Art Including Information Disclosed Under 37 CFR 1.97 and 1.98

The prior art relates to tapes used to prevent mouth breathing, such as for use as a sleep aid or anti-snore device. The prior art, as best understood, does not disclose a roll of anti-mouth-breathing tape that includes a spool and a strip of anti-mouth-breathing tape, wherein the strip includes a plurality of perforations oriented perpendicular to a length of the strip at intervals to form tape pieces that are selectively detachable from the strip when the strip is unwound.

### BRIEF SUMMARY OF THE INVENTION

An embodiment of the disclosure meets the needs presented above in a roll of anti-mouth-breathing tape generally comprising a spool and a strip of anti-mouth-breathing tape. The spool includes a spindle and the strip is wound about the spindle.

The strip includes a substrate layer and an adhesive layer on the substrate layer. The strip is elongated. The strip includes a plurality of perforations oriented perpendicular to a length of the strip. Each of the perforations is spaced apart at intervals to form tape pieces that are selectively detachable from the strip when the strip is unwound. Each of the tape pieces is designed to be removably attached to lips of a user to restrict breathing through a mouth of the user and promote breathing through a nose of the user.

An embodiment of the disclosure also relates to a method of restricting breathing through the mouth of the user using the toll of anti-mouth-breathing tape, which includes the steps of unrolling a tape piece of said tape pieces, tearing off the tape piece from the strip at a respective one of the perforations, placing the adhesive layer against lips of the user, and pressing the substrate layer to adhere the tape piece to the lips of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a roll according to an embodiment of the disclosure.

FIG. 2 is a perspective view of an embodiment of the disclosure.

FIG. 3 is a perspective view of an embodiment of the disclosure in use.

FIG. 4 is a perspective view of an embodiment of the disclosure in use.

FIG. 5 is a perspective view of a first alternative embodiment of the disclosure. po FIG. 6 is a perspective view of a second alternative embodiment of the disclosure.

FIG. 7 is a perspective view of a third alternative embodiment of the disclosure. po FIG. 8 is a perspective view of a fourth alternative embodiment of the disclosure.

FIG. 9 is a perspective view of a fifth alternative embodiment of the disclosure. po FIG. 10 is a perspective view of a sixth alternative embodiment of the disclosure.

FIG. 11 is a perspective view of an embodiment of the disclosure.

### DETAILED DESCRIPTION OF THE INVENTION

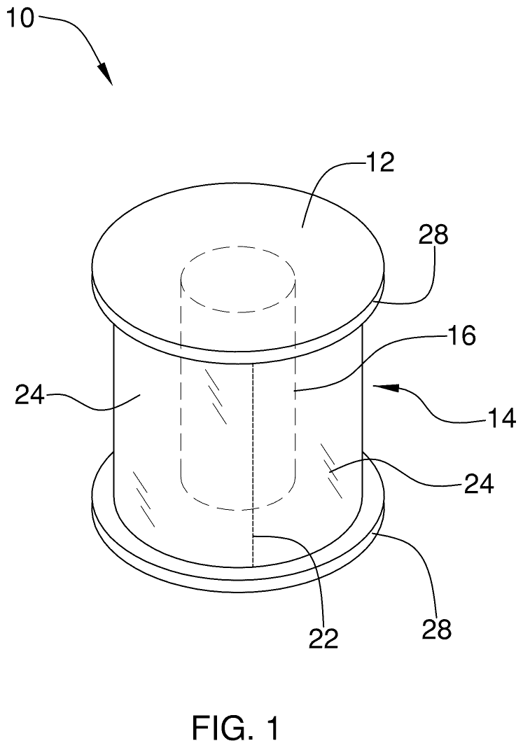

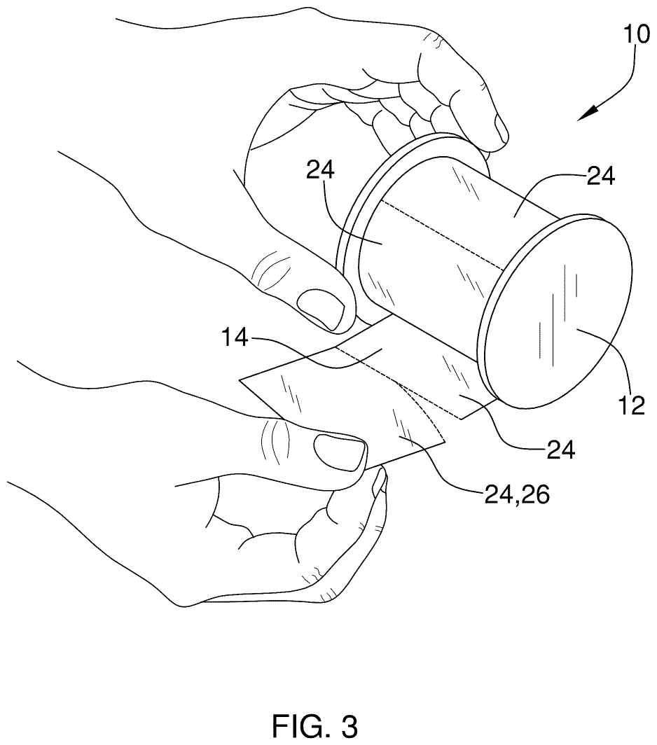

With reference now to the drawings, and in particular to FIGS. 1 through 11 thereof, a new roll embodying the principles and concepts of an embodiment of the disclosure and generally designated by the reference numeral 10 will be described.

As best illustrated in FIGS. 1 through 11, the roll 10 of anti-mouth-breathing tape generally comprises a spool 12 and a strip 14 of anti-mouth-breathing tape. The spool 12 includes a spindle 16 and the strip 14 is wound about the spindle 16. The strip includes a substrate layer 18 and an adhesive layer 20 on the substrate layer 18. The strip 14 is elongated. The strip 14 includes a plurality of perforations 22 oriented perpendicular to a length of the strip 14. Each of the perforations 22 is spaced apart at intervals to form tape pieces 24 that are selectively detachable from the strip 14 when the strip 14 is unwound. Each of the tape pieces 24 is designed to be removably attached to lips 72 of a user 70 to restrict breathing through a mouth 74 of the user 70 and promote breathing through a nose 76 of the user 70.

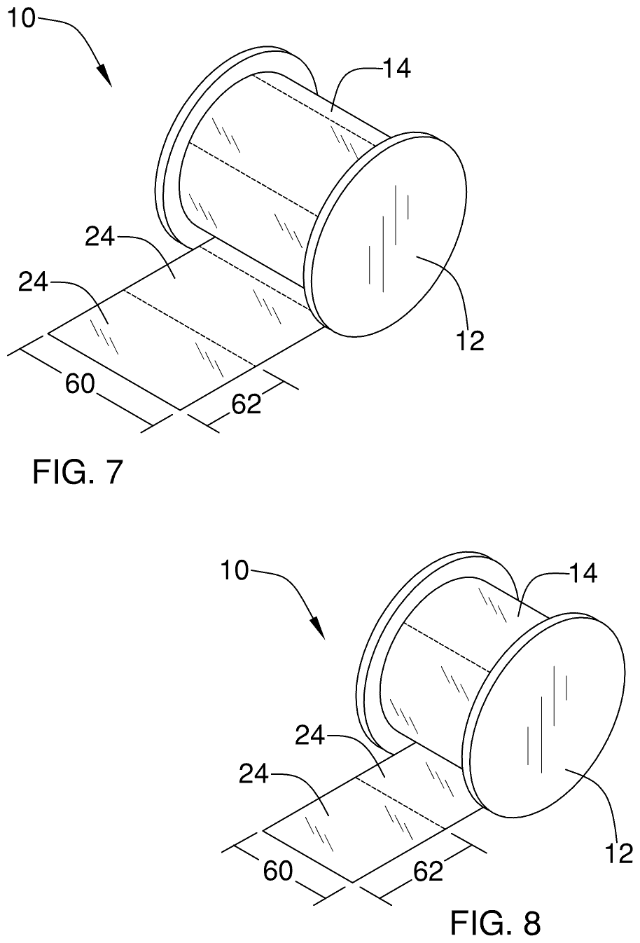

Each of the tape pieces 24 has a length dimension 60 and a width dimension 62 which are sized to fit the lips 72 of the user 70. For example, each of the tape pieces 24 could be sized to only cover a portion of the lips 72, such as a central portion, or possibly only the vermillion portion, that is, the red or pink skin of the lips 72. Each of the tape pieces 24 could also be sized to cover all or most all of the lips 72, including the vermillion and the outer skin extending toward the nose 76 on the upper lip and extending toward the chin on the lower lip, as well as lateral areas extending from the corners of the mouth 74 toward the cheeks. In one possible embodiment, the length dimension 60 is in the range of 25 mm to 100 mm, and the width dimension 62 is in the range of 10 mm to 70 mm, which ranges include all whole numbers within the ranges. In the exemplary embodiment in FIGS. 1 through 4, the length dimension 60 is somewhat larger than the width dimension 62, such as, for example, 50 mm by 35 mm. In the exemplary embodiment in FIG. 7, the length dimension 60 is about twice as large as the width dimension 62, such as, for example, 60 mm by 30 mm. In the exemplary embodiment in FIG. 8, the length dimension 60 is about or exactly the same as the width dimension 62, such as, for example, 40 mm by 40 mm.

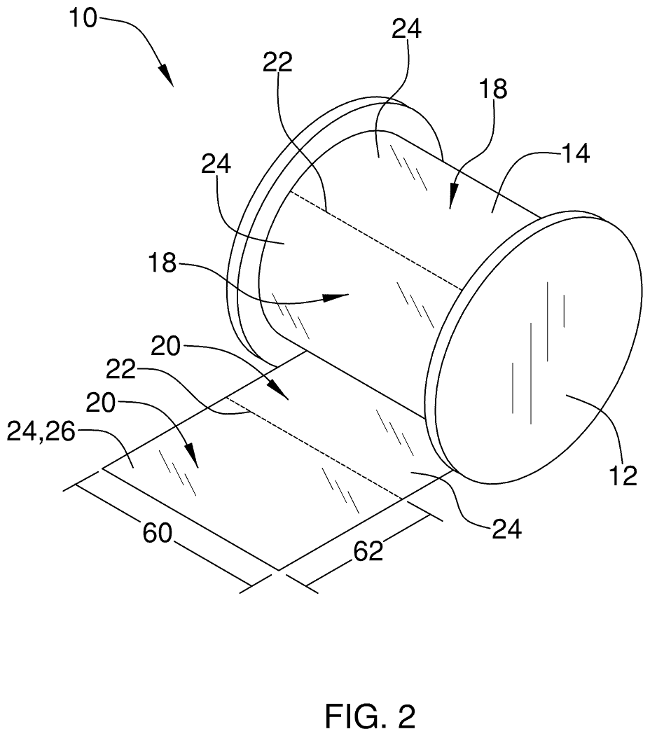

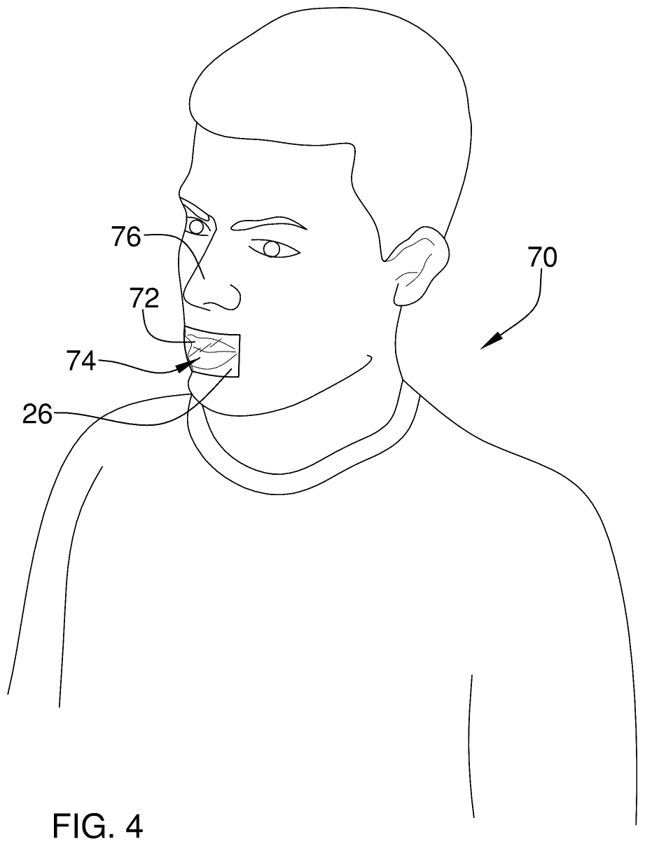

FIGS. 2 through 4 show how to use the roll 10 of anti-mouth-breathing tape to restrict breathing through the mouth 74 of the user 70. The user 70 first unrolls a tape piece 26 of the tape pieces 24. The user 70 then tears off the tape piece 26 from the strip 14 at a respective one of the perforations 22. While not shown, any of the tape pieces 24 that were unrolled or partially unrolled would be pressed back onto the strip 14. The user 70 then places the adhesive layer 20 against lips 72 of the user 70, and finally presses the substrate layer 18 to adhere the tape piece 26 to the lips 72 of the user 70 as shown in FIG. 4. When the user 70 is done using the tape piece 26, such as after sleeping, the user 70 can remove and discard the tape piece 26. This process can be repeated until all tape pieces 24 are used.

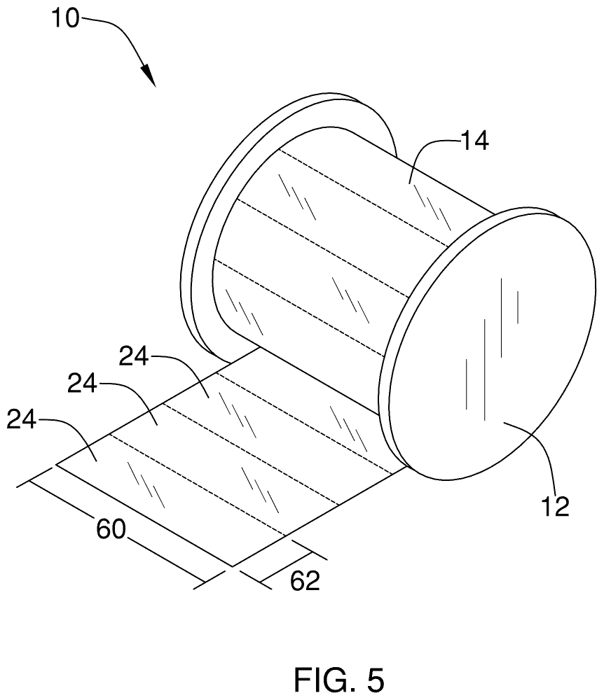

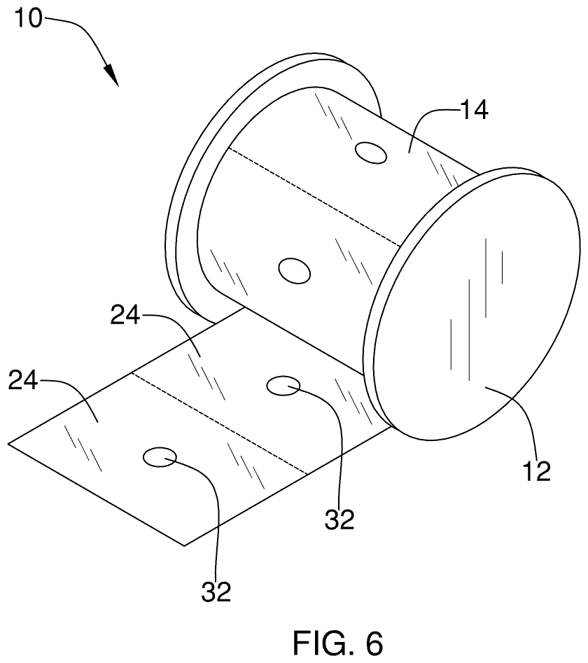

In one possible embodiment, the width dimension 62 could be outside the ranges mentioned above. In the exemplary embodiment shown in FIG. 5, the length dimension 60 of each of the tape pieces 24 is substantially greater than the width dimension 62, such as, for example, three, four, or five times. In this case, the length dimension 60 is in the range of 25 mm to 100 mm, but the width dimension 62 is in the range of 10 mm to 25 mm. For example, the length dimension 60 might be 50 mm and the width might be 10 mm. This particular design allows for customization by letting the user 70 unroll multiple tape pieces 24 to form a custom-length piece. For example, the user 70 could unroll a selected number of the tape pieces 24, such as three or four as shown in FIG. 5. The user 70 then tears off the last one of the selected number of the tape pieces 24 from the strip 14 at its respective one of the perforations 22, which thereby creates a custom-length piece consisting of the selected number of the tape pieces 24. The user 70 then proceeds in the same manner as shown in FIG. 4, that is, by placing the adhesive layer 20 of the custom-length piece against the lips 72 of the user 70, and then pressing the substrate layer 18 of the custom-length piece to adhere the custom-length piece to the lips 72 of the user 70.

Since the tape pieces 24 are used on the skin of the user 70 and placed on the lips 72 at the mouth 74, each of the substrate layer 18 and the adhesive layer 20 is made of hypoallergenic non-toxic materials. However, this is only one exemplary embodiment as the strip 14 could be made of different materials as desired or as would be appropriate for use on the skin and around the mouth 74. For example, the strip 14 could be medical-grade adhesive tape or biodegradable or eco-friendly tape. The substrate layer 18 could be made of various materials, such as, for example, silicone, hypoallergenic polyurethane, polyethylene or polypropylene film, latex-free elastic, plant-based polymers, thermoplastic elastomers (TPE), non-woven fabric, and breathable mesh or perforated material. The adhesive layer 20 could be made of gel-based adhesives, pressure-sensitive adhesive (PSA), removable adhesive that has low tack for sensitive skin, hydrogel adhesive that is moisture-activated, and biodegradable adhesive compounds.

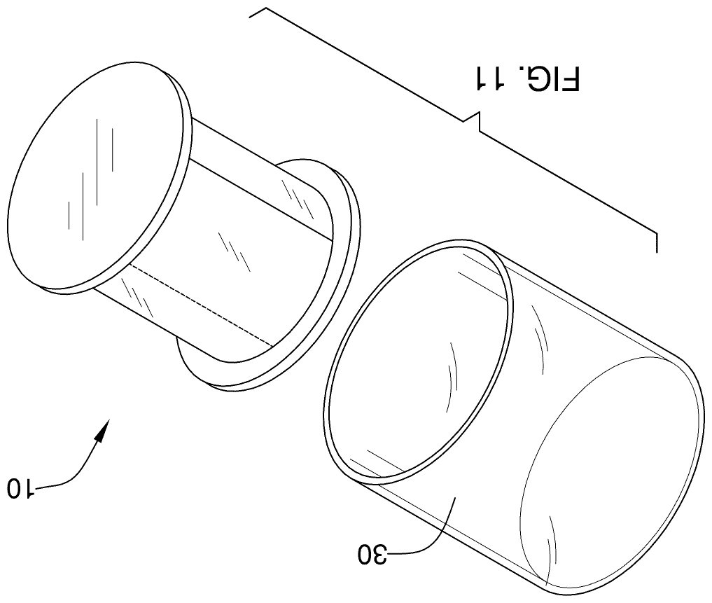

In the exemplary embodiment, the spool 12 includes a pair of rims 28 coupled at respective ends of the spindle 16. In another possible embodiment, the rims 28 could be replaced by rods or similar gripping structures that could extend from the spindle 16 to allow holding of the roll 10. FIG. 11 also shows a container 30 for storing the roll 10, which container 30 is open but could possibly include a cap.

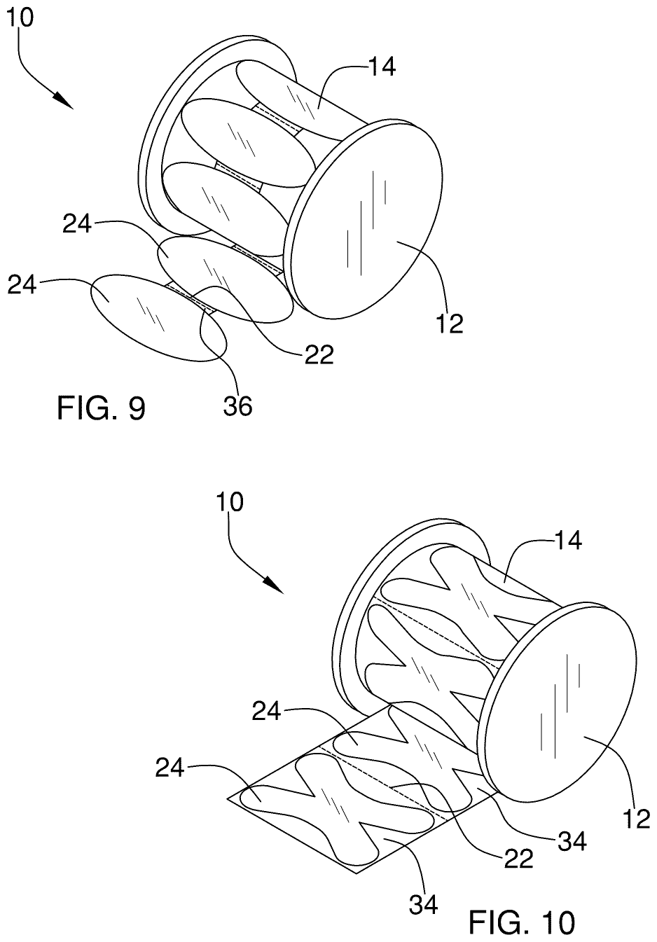

In the exemplary embodiment in FIGS. 1 through 7, each of the tape pieces 24 is rectangular, though this is only one possible design. In FIG. 8, the shape is a square. In FIG. 9, each of the tape pieces 24 is oval-shaped. As shown in FIG. 9, the tape pieces 24 are connected by a material web 36 that is perforated. Alternatively, these tape pieces 24 could be arranged lengthwise. In FIG. 10, each of the tape pieces 24 is X-shaped. However, these are just a few examples, and other possible shapes are circles, crescents for an ergonomic fit on the lips 72 and the mouth 74, combination shapes such as dumbbell-like shapes that have straight and curved portions, or essentially any kind of customizable or freeform shape. In addition, any of these shapes could include a central aperture 32, as shown in FIG. 6, that is designed for partial mouth 74 breathing, or possibly to allow drinking, such as through a straw.

For any of the shapes, such as the X-shape as shown in FIG. 10, the strip 14 further includes a backing layer 34 coupled to the adhesive layer 20. The backing layer 34 includes the perforations 22. In use, the user 70 performs essentially the same steps as described above, but in this case the step of tearing off the tape piece 26 includes tearing off a section of the backing layer 34 at a respective one of the perforations 22 in the backing layer 34. The user 70 then removes the tape piece 26 from the backing layer 34.

In one possible embodiment, the substrate layer 18 could be transparent or colored. The strip 14 could also include a scent-producing material or chemical to produce a desired scent, such as, for example, lavender, which is often used as a sleep aid. The substrate layer 18 could also be textured for enhanced grip.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A roll of anti-mouth-breathing tape comprising:
a spool including a spindle; and
a strip of anti-mouth-breathing tape being wound about said spindle, said strip including a substrate layer and an adhesive layer on said substrate layer, said strip being elongated, said strip including a plurality of perforations oriented perpendicular to a length of said strip, each of said perforations being spaced apart at intervals to form tape pieces being selectively detachable from said strip when said strip is unwound, each of said tape pieces being configured to be removably attached to lips of a user to restrict breathing through a mouth of the user and promote breathing through a nose of the user.

2. The roll of anti-mouth-breathing tape of claim 1, wherein each of said tape pieces has a length dimension and a width dimension, wherein said length dimension is in the range of 25 mm to 100 mm, and said width dimension is in the range of 10 mm to 70 mm.

3. The roll of anti-mouth-breathing tape of claim 1, wherein each of said substrate layer and said adhesive layer is made of hypoallergenic non-toxic materials.

4. The roll of anti-mouth-breathing tape of claim 1, wherein said spool includes a pair of rims coupled at respective ends of said spindle.

5. The roll of anti-mouth-breathing tape of claim 1, wherein each of said tape pieces is rectangular.

6. The roll of anti-mouth-breathing tape of claim 5, wherein each of said tape pieces has a length dimension and a width dimension, wherein said length dimension is in the range of 25 mm to 100 mm, and said width dimension is in the range of 10 mm to 25 mm.

7. The roll of anti-mouth-breathing tape of claim 1, wherein each of said tape pieces includes a central aperture configured for partial mouth breathing.

8. The roll of anti-mouth-breathing tape of claim 1, wherein each of said tape pieces is oval-shaped.

9. The roll of anti-mouth-breathing tape of claim 1, wherein each of said tape pieces is X-shaped.

10. The roll of anti-mouth-breathing tape of claim 1, wherein:
said strip further includes a backing layer coupled to said adhesive layer; and
said backing layer includes said perforations.

11. The roll of anti-mouth-breathing tape of claim 2, wherein each of said tape pieces is rectangular.

12. The roll of anti-mouth-breathing tape of claim 11, wherein:
said strip further includes a backing layer coupled to said adhesive layer; and
said backing layer includes said perforations.

13. The roll of anti-mouth-breathing tape of claim 2, wherein each of said tape pieces is oval-shaped.

14. The roll of anti-mouth-breathing tape of claim 13, wherein:
said strip further includes a backing layer coupled to said adhesive layer; and
said backing layer includes said perforations.

15. The roll of anti-mouth-breathing tape of claim 2, wherein each of said tape pieces is X-shaped.

16. The roll of anti-mouth-breathing tape of claim 15, wherein:
said strip further includes a backing layer coupled to said adhesive layer; and
said backing layer includes said perforations.

17. A method of restricting breathing through a mouth of a user using anti-mouth-breathing tape, said method comprising the steps of:
providing a roll of anti-mouth-breathing tape comprising:
a spool including a spindle; and
a strip of anti-mouth-breathing tape being wound about said spindle, said strip including a substrate layer and an adhesive layer on said substrate layer, said strip being elongated, said strip including a plurality of perforations oriented perpendicular to a length of said strip, each of said perforations being spaced apart at intervals to form tape pieces being selectively detachable from said strip when said strip is unwound, each of said tape pieces being configured to be removably attached to lips of a user to restrict breathing through a mouth of the user and promote breathing through a nose of the user;
unrolling a tape piece of said tape pieces;
tearing off said tape piece from said strip at a respective one of said perforations;
placing said adhesive layer against lips of the user; and
pressing said substrate layer to adhere said tape piece to the lips of the user.

18. The method of claim 17, wherein:
said strip further includes a backing layer coupled to said adhesive layer;
said backing layer includes said perforations;
said step of tearing off said tape piece includes tearing off a section of said backing layer at a respective one of said perforations in said backing layer; and
said method further comprises removing said tape piece from said backing layer.

19. A method of restricting breathing through a mouth of a user using anti-mouth-breathing tape, said method comprising the steps of:
providing a roll of anti-mouth-breathing tape comprising:
a spool including a spindle; and
a strip of anti-mouth-breathing tape being wound about said spindle, said strip including a substrate layer and an adhesive layer on said substrate layer, said strip being elongated, said strip including a plurality of perforations oriented perpendicular to a length of said strip, each of said perforations being spaced apart at intervals to form tape pieces being selectively detachable from said strip when said strip is unwound, each of said tape pieces being configured to be removably attached to lips of a user to restrict breathing through a mouth of the user and promote breathing through a nose of the user;
unrolling a selected number of said tape pieces;
tearing off a last one of said selected number of said tape pieces from said strip at a respective one of said perforations and thereby creating a custom-length piece consisting of said selected number of said tape pieces;
placing said adhesive layer of said custom-length piece against lips of the user; and
pressing said substrate layer of said custom-length piece to adhere said custom-length piece to the lips of the user.
